(19) United States
(12) Patent Application Publication
(10) Pub. No.: US 2019/0160002 A1
(43) Pub. Date: May 30, 2019

(54) PROCESS FOR PREPARING STERILE ARIPIPRAZOLE FORMULATION

(71) Applicant: Mylan Laboratories Ltd., Hyderabad (IN)

(72) Inventors: Bhaveshkumar Vallabhbhai Patel, Hyderabad (IN); Manoj Pananchukunnath, Hyderabad (IN); Arvindrao Atul Bodkhe, Hyderabad (IN)

(73) Assignee: Mylan Laboratories Limited, Hyderabad (IN)

(21) Appl. No.: 16/320,713

(22) PCT Filed: Jul. 28, 2017
(86) PCT No.: PCT/IB2017/001104

§ 371 Date: Jan. 25, 2019

(30) Foreign Application Priority Data
Jul. 28, 2016 (IN) 201641025857

(51) Int. Cl. A61K 9/00; A61K 31/496; A61K 9/10; A61K 9/19; A61K 47/38; A61K 47/26; A61K 47/02

(52) U.S. Cl. CPC A61K 9/0019; A61K 31/496; A61K 9/10; A61K 47/02; A61K 47/38; A61K 47/26; A61K 9/19

(57) ABSTRACT

The invention provides a process for preparing a sterile suspension comprising aripiprazole. More particularly, the invention provides the process for preparing the sterile suspension comprising aripiprazole, wherein said process involves in-process moist-heat sterilization of said suspension comprising aripiprazole. The in-process moist-heat sterilization is particularly efficient and economical process for the sterilization of aripiprazole formulations for parenteral administration, wherein the formulation obtained by said process have comparable physicochemical characteristics with the commercially available Ability Maintena® Injection.

### FIELD OF INVENTION

The invention relates to a process for preparing sterile formulation comprising antipsychotic agent. More particularly, the invention relates to a process for preparing sterile formulation comprising aripiprazole and one or more other pharmaceutically acceptable excipient(s).

### BACKGROUND OF THE INVENTION AND RELATED PRIOR ART

Aripiprazole is an atypical antipsychotic agent, used for the treatment of schizophrenia. Chemically it is known as 7-[4-[4-(2,3-dichlorophenyl)-1-piperazinyl] butoxy]-3, 4 dihydrocarbostyril and is disclosed in U.S. Pat. No. 5,006,528.

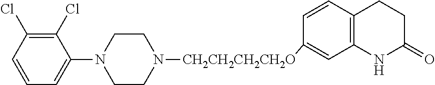

Aripiprazole is marketed in the U.S., under the trade name Abilify® in the form of tablet. It is also available as intramuscular extended release injection under the trade name Abilify Maintena Kit® and Abilify Maintena® in the U.S. and Europe respectively.

Ability Maintena® or Abilify Maintena Kit® is marketed as sterile aripiprazole aqueous suspension. In particular, the suspension is obtained by suspending sterile active pharmaceutical ingredient (API), aripiprazole monohydrate with a mean particle size of about 1 μm to about 10 μm in an aqueous vehicle. It is available as lyophilized powder and dispensed either in single dose vial or in pre-tilled dual chamber syringe.

For any parenteral preparation, sterilization is one of the essential feature, since said preparation is introduced directly into the human body. Sterilization is carried out mainly in two ways. One way is where the bulk API is first sterilized and then said sterile API is formulated under aseptic conditions, while the other way is to formulate unsterile bulk API and then the final formulation, packed in the desired container is terminally sterilized. The selection of a sterilization process is mainly based upon the physicochemical properties of drug as well as type of formulation i.e. liquid or powder or containers such as prefilled syringe or cartridge.

Various studies found that aripiprazole monohydrate is discolored and decomposed by both gamma-ray and electron beam, and is melted by dry heat sterilization. It is also seen that aripiprazole monohydrate particles in aqueous suspension tend to agglomerate by steam heat sterilization. Further, it is also found that aripiprazole is not suitable for terminal sterilization. Hence, aseptic processing by using sterile aripiprazole monohydrate, instead of terminal sterilization, is employed for preparation of Abilify Maintena® injection (Ref.: Abilify Maintena: EPAR—Public assessment report. EMEA published on Nov. 28, 2013).

Patents/published patent applications, which disclose sterile aripiprazole preparation or parenteral extended release preparation comprising sterile aripiprazole API followed by aseptic processing, are disclosed below:

U.S. Pat. No. 9,066,848 discloses process for preparing sterile crystals of aripiprazole monohydrate of desired small particle size and narrow particle size distribution by jet stream without milling.

U.S. Pat. Nos. 7,807,680; 8,030,313 and 8,722,679 discloses method for preparing sterile freeze-dried formulation comprising the steps of:

(a) preparing sterile bulk aripiprazole having a desired particle size distribution;

(b) preparing a sterile vehicle for the sterile bulk aripiprazole;

(c) combining the sterile aripiprazole and the sterile vehicle to form a sterile primary suspension that includes a sterile mixture of solids;

(d) reducing the mean particle size of the sterile mixture of solids in the sterile primary suspension e.g., by an aseptic wet milling to within the range from about 1 μm to about 100 μm particularly about 1 μm to 10 μm, to form a sterile final suspension; and finally

(e) freeze-drying the sterile final suspension to form the sterile freeze-dried formulation.

US published patent application no. 2010/0196486 discloses a method for preparing aripiprazole suspension comprising the steps of:

(I) combining sterile bulk aripiprazole with a mean particle size of 200 μm to 400 μm and a sterile vehicle to form a sterile primary suspension;

(II) subjecting the sterile primary suspension to first pulverization using a high shear pulverizing machine or a dispersion machine that applies shear force to a material to be processed, to form a sterile secondary suspension; and

(III) subjecting the sterile secondary suspension to second pulverization using a high-pressure homogenizer to form a sterile final suspension; wherein the aripiprazole in the sterile final suspension has a mean particle size of 1 μm to 10 μm.

US published patent application no. 2014/0112993 discloses freeze-dried aripiprazole formulation and water for injection separately, which are mixed together immediately before use to reconstitute a ready-to-use suspension. It further discloses that the aripiprazole used for this formulation is mainly comprising sterile bulk aripiprazole having desired particle size distribution.

The above prior arts disclose various formulation/process for preparing sterile aripiprazole suspension by using sterile bulk aripiprazole in aseptic processing which may be prepared by using either jet stream crystallization or milling or pulverization etc. However, the sterilization process for the bulk API is costly, cumbersome and involves complete sterile logistics, transportation and chain log, which is many times difficult to maintain. Hence, there is a need to develop an improved process for formulating an API, which is simple, cost effective and eliminate the burden of complexity involved with the sterile API, as discussed above.

The inventors of the present invention developed sterile aripiprazole suspension or the formulation comprising the same, by using non-sterile bulk aripiprazole as the starting material, where sterilization is achieved during the process itself. In particular, the process is designed to include in-process moist heat sterilization step for the preparation of aripiprazole suspension, which is simple, cost effective and moreover, the suspension prepared by said process have comparable physicochemical parameters against the commercially available Abilify Maintena® Injection.

### SUMMARY AND OBJECTIVES OF THE INVENTION

The invention relates to a process for preparing a sterile suspension comprising aripiprazole.

In particular, the invention relates to a process for preparing a sterile suspension comprising aripiprazole, wherein said process involves in-process moist-heat sterilization of said suspension comprising aripiprazole.

More particularly, the invention further relates to a process for preparing a sterile suspension comprising aripiprazole, at least one viscosity enhancing agent and one or more other pharmaceutically acceptable excipient(s), wherein said process involves in-process moist-heat sterilization of said suspension comprising aripiprazole.

The invention further relates to a composition comprising sterile suspension of aripiprazole, at least one viscosity enhancing agent and one or more other pharmaceutically acceptable excipient(s), wherein said composition is prepared by a process which involves in-process moist-heat sterilization of said suspension comprising aripiprazole.

The invention also relates to a composition comprising sterile suspension of aripiprazole, at least one viscosity enhancing agent and one or more other pharmaceutically acceptable excipient(s), wherein said composition is prepared by a process which involves in-process moist-heat sterilization of said suspension comprising aripiprazole having comparable physicochemical parameters with the commercially available Abilify Maintena® Injection.

Yet in another aspect, the present invention relates to a process for preparing a sterile suspension comprising aripiprazole, wherein said process does not involves the use of terminal sterilization of said suspension comprising aripiprazole.

### DETAILED DESCRIPTION OF THE EMBODIMENTS OF THE INVENTION

The invention relates to a process for preparing a sterile suspension comprising aripiprazole. More particularly, the invention relates to a process for preparing a sterile suspension comprising aripiprazole, wherein said process involves in-process moist-heat sterilization of said suspension comprising aripiprazole.

The invention also relates to a composition comprising sterile suspension of aripiprazole, at least one viscosity enhancing agent and one or more other pharmaceutically acceptable excipient(s), wherein the composition is prepared by in-process moist-heat sterilization of said suspension comprising aripiprazole.

More particularly, the invention relates to a composition comprising sterile suspension of aripiprazole, at least one viscosity enhancing agent and one or more other pharmaceutically acceptable excipient(s), wherein the composition is prepared by using non-sterile bulk aripiprazole as the starting material, wherein moist-heat sterilization is done in-process.

The term “in process moist-heat sterilization” as per present invention refers to the moist-heat sterilization which is conducted or has been achieved during the process of preparation of formulation comprising aripiprazole.

The invention also relates to a process for preparing a sterile suspension comprising aripiprazole, wherein said process does not involves the use of terminal sterilization of said suspension comprising aripiprazole.

According to the Food and Drug Administration's Center for Drug Evaluation and Research Data Standards Manual (CDER Data Element Number C-DRG-00301; Data Element Name: Route of Administration), the term “parenteral” refers to administration by injection, infusion or implantation. The parenteral formulation according to the invention relates to the intramuscular or subcutaneous injection and preferably to the intramuscular injection.

The term “sterile suspension” according to the invention relates to the suspension used for parenteral administration which is free from all viable microbial contamination.

The term “terminal sterilization” according to the invention relates to the final or end-process sterilization where the final formulation is prepared, packed in the desired container and then the container is subjected to sterilization step.

Moist heat sterilization is the widely used method for the sterilization of surgical dressings, sheets, surgical and diagnostic equipment, containers, closures, aqueous injections, ophthalmic preparations and irrigation fluids etc. It mainly involves the use of steam in the range of 121-134° C. Steam under pressure is used to generate high temperature needed for sterilization.

The aripiprazole present in the sterile composition is in an amount within the range from about 8% to about 12% (w/v), based on the total composition.

Viscosity enhancing agents is present in an amount within the range from about 0.2% to about 6% by weight, based on the total weight of the sterile composition. These agents when added to the solution/suspension increases its viscosity without substantially modifying other properties and hence increase the stability and pourability. Non-limiting examples of such viscosity enhancing agent(s) includes methylcellulose, hydroxyethylcellulose, hydroxypropylcellulose, sodium carboxymethyl cellulose, polyvinylpyrrolidone, sodium alginate and the like or combinations thereof. More preferably, the viscosity enhancing agent used according to the present invention is sodium carboxymethyl cellulose.

The parenteral compositions comprising aripiprazole according to present invention further includes one or more other pharmaceutically acceptable excipient(s), which may be selected from a group comprising of bulking agent(s), buffering agent(s) and/or pH adjusting agent(s).

Bulking agent is present in an amount within the range from about 1% to about 3% by weight based on the total weight of the sterile composition. Bulking agents according to the invention, are selected from a group comprising of mannitol, sucrose, maltose, xylitol, glucose, starches, sorbitol and the like or combinations thereof.

Buffering agents is present in an amount within the range from about 0.02% to about 0.05% by weight based on the total weight of the sterile composition. Buffering agents according to the invention, are selected from a group comprising phosphate, acetate, succinate, tartarate, ascorbate, citrate, lactate, sodium phosphate, potassium phosphate, sodium dihydrogen phosphate monohydrate (sodium phosphate monobasic monohydrate) and the like or combinations thereof.

The composition of the present invention may optionally include a pH adjusting agent(s) which is employed in amount sufficient to adjust pH of the composition within the range from about 6 to about 7.5, preferably about 7. The pH adjusting agents according to the invention, are selected from a group comprising sodium hydroxide, potassium hydroxide and the like or combinations thereof.

The moist heat sterilization process according to the invention can be carried out in an autoclave which uses moist steam to sterilize the composition at about 121° C.-134° C. for at least 20 minutes. In one embodiment, moist heat sterilization may be performed by using sterilization in place (SIP) system. This step may also be referred to as steam sterilization, according to the embodiments of the invention.

Yet in another embodiment, aripiprazole used for the preparation of sterile aripiprazole suspension may be either anhydrous or hydrous in various stage of hydration. More preferably, the present invention used aripiprazole monohydrate.

In an embodiment, the sterilized suspension obtained in the process further undergo homogenization and milling to get the desired particle size.

The term “Homogenization” as per the present invention refers to the process which is used to prepare a uniform sterile aripiprazole suspension which may done by using either Mechanical or Ultrasonic process.

The term “Milling” as per the present invention refers to the process which is used to prepare uniform particle size of sterile aripiprazole suspension which may be done using bead mill, ball mill, roller mill, Netzsch mill, DC mill or planetary mill. However, it is essential that the milling procedure and equipment employed to get the desired mean particle size of aripiprazole particle in sterile suspension or formulation.

The term “Lyophilization” as per the present invention refers to the process in which the composition is rapidly frozen and dehydrated under high vacuum. Technically, lyophilization is also known as lyophilisation or freeze-drying or cryodesiccation. Lyophilization typically includes the steps of pretreatment, freezing, primary drying and secondary drying. Methods for lyophilizing, the compositions are known (Ref: U.S. Pat. No. 6,199,297) and routinely used.

The term “mean particle size” as per the present invention refers to the volume mean diameter or D [4,3.] The particle size distribution is expressed in terms of D10, D50, and D90. The volume mean diameter (expressed in microns) and particle size distribution (expressed in microns) were measured by laser diffraction technique using a Malvern Mastersizer 2000 instrument. Specific surface area (expressed in m²/g) was also measured using Malvern Mastersizer 2000.

According to an embodiment of the present invention, the invention provides a process for preparing a sterile suspension comprising aripiprazole, wherein said process involves the steps of:
a. preparing a suspension by mixing aripiprazole, at least one viscosity enhancing agent and one or more other pharmaceutically acceptable excipient(s) in purified water,
b. sterilizing, the suspension of step (a),
c. homogenizing the sterilized suspension of step (b), and
d. milling the homogenized suspension of step (c).
   wherein the sterilization of step (b) is done by moist-heat sterilization.

According to an embodiment, the invention also relates to a process for preparing a sterile suspension comprising aripiprazole for parenteral administration involving the steps of:
a. preparing a suspension by mixing aripiprazole, at least one viscosity enhancing agent and one or more other pharmaceutically acceptable excipient(s) in purified water,
b. sterilizing the suspension of step (a),
c. homogenizing the sterilized suspension of step (b),
d. milling the homogenized suspension of step (c), and
e. lyophilizing the milled suspension of step (d),
   wherein the sterilization of step (b) is clone by moist-heat sterilization.

Yet in another embodiment, the invention also relates to a composition comprising a sterile suspension of aripiprazole, wherein said composition is prepared by:
a. preparing a suspension by mixing aripiprazole, at least one viscosity enhancing agent and one or more other pharmaceutically acceptable excipient(s) in purified water,
b. sterilizing the suspension of step (a),
c. homogenizing the sterilized suspension of step (b),
d. milling the homogenized suspension of step (c), and
e. lyophilizing the milled suspension of step (d),

wherein the sterilization of step (b) is done by moist-heat sterilization.

Yet in another embodiment, the invention also relates to a composition comprising a sterile suspension of aripiprazole, wherein said composition is prepared by:
a. preparing a suspension by mixing aripiprazole, at least one viscosity enhancing agent and one or more other pharmaceutically acceptable excipient(s) in purified water,
b. sterilizing the suspension of step (a),
c. homogenizing the sterilized suspension of step (b),
d. milling the homogenized suspension of step (c) to get mean particle size of the aripiprazole in said sterile suspension within the range from about 1 to about 10 microns, and
e. lyophilizing the milled suspension of step (d),

wherein the sterilization of step (b) is done by moist-heat sterilization.

The parenteral composition is compounded or formulated before lyophilization by using either purified water, water for injection (WFI) or by using suitable solvent.

The sterile aripiprazole suspension of the invention, results in a single white lyophilized cake on lyophilization, which is easy to reconstitute to form a uniform, homogeneous suspension.

The present inventors surprisingly found that the sterile aripiprazole suspension prepared using in-process moist heat sterilization step, have same physicochemical characterization such as physical appearance of suspension, pH, osmolality, viscosity, specific surface area and content uniformity and particle size to that of commercially available Abilify Maintena® Injection.

Yet in another embodiment, there is provided a composition which can be distributed to the dispensing person, e.g., in a pharmacy or a hospital, in the form of a vial or pre-filled syringe containing the composition, or in the form of a kit comprising a vial or vials containing the composition and an appropriate amount of a suitable solvent.

The sterile lyophilized formulations of the invention may be reconstituted with an amount of water for injection to provide from about 1.0 to about 400 mg of aripiprazole delivered in a volume of 2.5 mL or less, preferably 2 mL for a two to six week dosage.

The sterile aripiprazole formulations of the invention may be used in the treatment of schizophrenia and related disorders such as dementia and bipolar disorder in human patients.

The following examples illustrate specific aspects and embodiments of the invention and demonstrate the practice and advantages thereof. It is to be understood that the examples are given by way of illustration only and are not intended to limit the scope of the invention in any manner.

### EXAMPLE 1

### Unit Composition:

| S. | | | | |
|---|---|---|---|---|
| No. | Ingredients | mg/vial | mg/vial | mg/mL |
| 1 | Aripiprazole | 416.06 | 312.04 | 104.02 |
| | monohydrate* | | | |
| 2 | Sodium carboxymethyl | 16.64 | 12.48 | 4.16 |
| | cellulose | | | |
| 3 | Sodium phosphate | 1.48 | 1.11 | 0.37 |
| | monobasic | | | |
| | monohydrate | | | |
| 4 | Mannitol | 83.20 | 62.40 | 20.80 |
| 5 | Sodium hydroxide | q.s to pH 7 | q.s to pH 7 | q.s to pH 7 |
| 6 | Purified water | q.s. to | q.s. to | q.s. to |
| | | 4 ml | 3 ml | 1 ml |

(*As used herein and hereafter, 416.06 mg, 312.04 and 104.02 mg of aripiprazole monohydrate are equivalent to 400 mg, 300 mg and 100 mg of aripiprazole respectively).

### Brief Manufacturing Process:

1. Purified water was taken in a glass beaker and heated to about 60° C.
2. To the heated water of step (1), sodium carboxymethyl cellulose was added under stirring followed by cooling till the mixture reaches to room temperature.

3. To the cooled mixture of step (2), sodium phosphate monobasic monohydrate and mannitol were added followed by mixing. The pH of the resultant mixture was adjusted to about 7.0 using 0.1 N Sodium Hydroxide
4. The volume of the mixture of step (3) was made with water followed by filtering the mixture through 0.2 μm filter.
5. To the mixture of step (4), dispensed quantity of aripiprazole monohydrate was added under stirring using magnetic stirrer to get a suspension, which was then sterilized by moist heat sterilization process carried out at 121° C. for 20 minutes followed by cooling to get the sterilized suspension.
6. The sterilized suspension of step (5) was homogenized followed by high speed bead milling to get uniform aripiprazole suspension.
7. The volume of the suspension of step (6) was adjusted to 100% of total batch size with purified water.

### EXAMPLE 2

### Unit Composition:

| S. | | | | |
|---|---|---|---|---|
| No. | Ingredients | mg/vial | mg/vial | mg/mL |
| 1 | Aripiprazole | 416.06 | 312.04 | 104.02 |
| | monohydrate | | | |
| 2 | Sodium carboxymethyl | 16.64 | 12.48 | 4.16 |
| | cellulose | | | |
| 3 | Sodium phosphate | 1.48 | 1.11 | 0.37 |
| | monobasic | | | |
| | monohydrate | | | |
| 4 | Mannitol | 83.20 | 62.40 | 20.80 |
| 5 | Sodium hydroxide | q.s to pH 7 | q.s to pH 7 | q.s. to pH 7 |
| 6 | Purified water* | Q.S to 4 ml | Q.S. to 3 ml | Q.S to 1 ml |

*Removed during lyophilization step

### Brief Manufacturing Process:

1. Purified water was taken in a glass beaker and heated to about 60° C.
2. To the heated water of step (1), sodium carboxymethyl cellulose was added under stirring followed by cooling till the mixture reaches to room temperature.
3. To the cooled mixture of step (2), sodium phosphate monobasic monohydrate and mannitol were added followed by mixing. The pH of the resultant mixture was adjusted to about 7.0 using 0.1 N Sodium Hydroxide
4. The volume of the mixture of step (3) was made with water followed by filtering the mixture through 0.2 μm filter.
5. To the mixture of step (4), dispensed quantity of aripiprazole monohydrate was added under stirring using magnetic stirrer to get a suspension (slurry) which was then sterilized by moist heat sterilization process carried out at 121° C. for 20 minutes followed by cooling to get the sterilized suspension.
6. The sterilized suspension of step (5) was homogenized followed by high speed bead milling to get uniform aripiprazole suspension.
7. The volume of the suspension of step (6) was adjusted to 100% of total batch size with purified water.
8. The suspension of step (7) was filled in a glass vials under continuous stirring and the vials were half stoppered with stoppers.
9. The vials were then subjected to lyophilization according to stages given below:
   a) Freezing was carried out in three cycles using the parameters as provided below:

| | Freezing cycle | | |
|---|---|---|---|
| | 1 | 2 | 3 |
| Temperature (in ° C.) | −5 | −45 | −45 |
| Time (in minutes) | 30 | 120 | 180 |

b) Primary drying was carried out in five cycles after completion of freezing using the parameters as provided below:

| | Primary drying cycles | | | | |
|---|---|---|---|---|---|
| | 1 | 2 | 3 | 4 | 5 |
| Temperature (in ° C.) | −10 | −10 | −5 | −5 | 0 |
| Time (in minutes) | 120 | 600 | 60 | 300 | 60 |
| Vacuum (mtorr) | 300 | 300 | 300 | 300 | 300 |

c) Secondary drying was carried out in two cycles after completion of primary drying using the parameters as provided below:

Secondary
drying cycles

| | 1 | 2 |
|---|---|---|
| Temperature (in ° C.) | 0 | 25 |
| Time (in minutes) | 600 | 120 |
| Vacuum (in mtorr) | 300 | 300 |

10. After lyophilisation, the vials were stoppered, unloaded from lyophilizer, sealed and labelled.

### Sterility Testing:

The compositions prepared in accordance with Examples 1 and 2 were subjected to sterility test as per United States Pharmacopoeia (USP), chapter <71> (“USP <71>”) and the growth of microorganisms, if any, was observed.
The sterility testing was done by membrane-filtration method using below procedure:
I. Cellulose membrane (0.45 micron) was pre-wetted with 50 mL of 0.1% peptone.
II. Sample A (chosen from compositions of example 1) and Sample B (prepared by mixing 1.9 mL of 0.1% peptone to sample vial of compositions of example 2) was filtered through the membrane.
III. The membrane was rinsed with 3×100 mL volumes of 0.1% Peptone.
IV. The membrane was then cut into two equal parts and half of the membrane was transferred to Soya-bean casein digest medium (SCDM) while another half of the membrane was transferred to Fluid Thioglycolate medium (FTM).
V. The SCDM and FTM tubes were kept for incubation at 20-25° C. and 30-35° C. respectively till 14 days.
VI. Diluent control (0.1% peptone), Media controls were also incubated for 14 days.
   The results of sterility test are shows in Table 1:

Table 1:

### Sterility Test Results:

| | Test | | |
|---|---|---|---|
| Sample | parameter | Requirements | Results |
| Sample A | Sterility | shall not show any evidence of | Complies test |
| | test | microbial growth after 14 days | as per USP |
| | | of incubation period. | |
| Sample B | Sterility | shall not show any evidence of | Complies test |
| | test | microbial growth after 14 days | as per USP |
| | | of incubation period. | |

It can be seen from the results provided in Table 1 that no growth of microorganisms was observed after 14 days of incubation period. The results thus indicate that the compositions of present invention were meeting the sterility requirements per USP chapter <71>.

Table 2:

### Particle Size Distribution of Aripiprazole in Manufacturing Stages:

| | Particle Size | | |
|---|---|---|---|
| | Distribution (in μm) | | |
| Manufacturing stages of aripiprazole | of aripiprazole | | |
| formulation | D10 | D50 | D90 |
| Pre-sterilised suspension | 3.156 | 11.897 | 76.445 |
| (Before Moist heat sterilization) | | | |
| Post-sterilization suspension | 5.670 | 19.434 | 126.144 |
| (Moist heat sterilized slurry) | | | |
| Pre-milled suspension | 3.701 | 7.132 | 13.038 |
| (Homogenized slurry) | | | |
| Post-milled suspension | 1.702 | 3.373 | 7.010 |
| Reconstituted suspension obtained by | 1.779 | 3.357 | 6.647 |
| suspending lyophilised cake in WFI | | | |

D10 = particle size, 10% of particles lower than given value
D50 = particle size, 50% of particles lower than given value
D90 = particle size, 90% of particles lower than given value

The results as provided in Table 2 indicates that there was agglomeration of aripiprazole particles after moist heat sterilization. This was overcome by homogenizing the steam sterilized suspension using a homogenizer (Polytron homogenizer). The homogenized suspension was further milled using high speed bead mill (Netzsch DV15-300 mill) to get the desired mean particle size of aripiprazole.

### Comparative Physicochemical Characterization:

TABLE 3

| | Example 1 and | Abilify Maintena ® |
|---|---|---|
| Physicochemical parameters | Example 2 | Injection |
| Description of lyophilised | White lyophilized | White lyophilized |
| formulation | cake | cake |
| Description of reconstituted | Opaque and milky- | Opaque and milky- |
| suspension | white uniform, | white uniform, |
| | homogeneous | homogeneous |
| | suspension | suspension |
| pH of reconstituted | 7.05 | 7.00 |
| suspension | | |
| Osmolality | 315 | 321 |
| Viscosity (Cps) | 8.524 | 8.2623 |
| Particle Size | | |
| (expressed in μm) | | |
| D10 | 1.693 | 1.542 |
| D50 | 3.051 | 3.527 |
| D90 | 5.641 | 9.202 |
| D (4,3) | 3.413 | 4.592 |
| Assay | 458.4 | 471.8 |
| G impurity | 0.07 | 0.04 |
| F impurity | ND | ND |
| Bis impurity | ND | ND |
| Dimer impurity | ND | ND |
| 4,4 Dimer impurity | ND | ND |
| Single Unknown max | 0.02 | 0.02 |
| impurity | | |
| Total Impurity | 0.11 | 0.06 |

As used herein, the “ND” wherever appears is an abbreviation for “Not detected”

The assay and impurity profiling data was obtained using Agilent HPLC system.

It can be seen from the physicochemical parameters provided in Table 3 that formulations prepared according to the present invention was found to be within specifications and comparable with the Abilify Maintena® Injection.

We claim:

1. A process for preparing a sterile suspension comprising aripiprazole, wherein said process involves the steps of:
a. preparing a suspension by mixing aripiprazole, at least one viscosity enhancing agent and one or more other pharmaceutically acceptable excipient(s) in purified water,
b. sterilizing the suspension of step (a),
c. homogenizing the sterilized suspension of step (b), and
d. milling the homogenized suspension of step (c),
wherein the sterilization of step (b) is done by moist-heat sterilization at 121° C. for at least 20 minutes.

2. A process for preparing a sterile composition comprising aripiprazole for parenteral administration involving the steps of:
a. preparing a suspension by mixing aripiprazole, at least one viscosity enhancing agent and one or more other pharmaceutically acceptable excipient(s) in purified water,
b. sterilizing the suspension of step (a),
c. homogenizing the sterilized suspension of step (b),
d. milling the homogenized suspension of step (c), and
e. lyophilizing the milled suspension of step (d),
wherein the sterilization of step (b) is done by moist-heat sterilization at 121° C. for at least 20 minutes.

3. A composition comprising a sterile suspension of aripiprazole for parenteral administration, wherein said composition is prepared by:
a. preparing a suspension by mixing aripiprazole, at least one viscosity enhancing agent and one or more other pharmaceutically acceptable excipient(s) in purified water,
b. sterilizing the suspension of step (a),
c. homogenizing the sterilized suspension of step (b),
d. milling the homogenized suspension of step (c), and
e. lyophilizing the milled suspension of step (d),
wherein the sterilization of step (b) is done by moist-heat sterilization at 121° C. for at least 20 minutes.

4. The process or composition according to any of the claims 1-3, wherein said process or composition does not involves the use of terminal sterilization of said suspension comprising aripiprazole.

5. The process or composition according to any of the claims 1-3, wherein the aripiprazole used prior to moist heat sterilization is present in non-sterile form.

6. The process or composition according to any of the claims 1-3, wherein said viscosity enhancing agent is selected from methylcellulose, hydroxyethylcellulose, hydroxypropylcellulose, sodium carboxymethyl cellulose, polyvinylpyrrolidone, sodium alginate or combinations thereof.

7. The process or composition according to any of the claims 1-3, wherein said other excipient(s) are selected from a group comprising bulking agent(s), buffering agent(s) and/or pH adjusting agent(s).

8. The bulking agent(s) according to claim 7, is selected from mannitol, sucrose, maltose, xylitol, glucose, starches, sorbitol or combinations thereof.

9. The buffering agent(s) according to claim 7, is selected from phosphate, acetate, succinate, tartarate, ascorbate, citrate, lactate, sodium phosphate, sodium phosphate monobasic monohydrate, potassium phosphate or combinations thereof.

10. The pH adjusting agent(s) according to claim 7, is selected from sodium hydroxide, potassium hydroxide, magnesium oxide, magnesium hydroxide or combinations thereof.
